(19) United States
(12) Patent Application Publication
(10) Pub. No.: US 2023/0015028 A1
(43) Pub. Date: Jan. 19, 2023

(54) DIAGNOSING RESPIRATORY MALADIES FROM SUBJECT SOUNDS

(71) Applicant: ResApp Health Limited, Red Hill, QLD (AU)

(72) Inventors: Vesa Tuomas Kristian Peltonen, Red Hill, QLD (AU); Javan Tanner Wood, Red Hill, QLD (AU)

(21) Appl. No.: 17/757,543

(22) PCT Filed: Dec. 16, 2020
(86) PCT No.: PCT/AU2020/051382

§ 371 Date: Jun. 16, 2022

(30) Foreign Application Priority Data
Dec. 16, 2019 (AU) 2019904754

(51) Int. Cl. A61B 5/00; G10L 25/66; A61B 7/00; A61B 5/08

(52) U.S. Cl. CPC A61B 5/7275; G10L 25/66; A61B 7/003; A61B 5/7267; A61B 5/0823; A61B 5/7257

(57) ABSTRACT

A method for predicting the presence of a malady of the respiratory system in a subject comprising: operating at least one electronic processor to transform one or more sounds of the subject that are associated with the malady into corresponding one or more image representations of said sounds; applying said one or more representations to at least one pattern classifier trained to predict the presence of the malady; and operating said processor to predict the presence of the malady in the subject based on at least one output of the at least one pattern classifier.

### TECHNICAL FIELD

The present application claims priority from Australian provisional patent application No. 2019904754 filed 16 Dec. 2019, the disclosure of which is hereby incorporated herein by reference.

### TECHNICAL FIELD

The present invention relates to an apparatus and a method for processing subject sounds for diagnosis of respiratory maladies.

### BACKGROUND

Any references to methods, apparatus or documents of the prior art are not to be taken as constituting any evidence or admission that they formed, or form part of the common general knowledge.

It is known to electronically process subject sounds to identify respiratory maladies. One way in which such processing is commonly done is to extract features from segments of the sound that are associated with a malady in question. For example, the malady in question might be pneumonia in which case the associated segments of the sound are segments that comprise cough sounds of the subject. The features of the cough sound that are extracted are typically values that quantify various properties of segments of the sound. For example, the number of zero crossings in the time domain of a segment of the cough sound waveform may be one feature. Another feature may be a value indicating deviation from Gaussian distribution of a segment of the cough sound. Other features may be logarithm of energy level for segments of the cough sound.

Once the values for the features have been determined, they are formed into a feature vector. Feature vectors for cough sounds from subjects known to be suffering, or not suffering, from a particular malady are then used as training vectors to train a pattern classifier such as a neural network. The trained classifier can then be used to classify a test feature vector as either being very likely to be predictive that the subject is suffering from the particular malady or not.

It will therefore be realized that such machine learning based, automatic diagnosis systems are very helpful. Indeed, it is possible to configure a processor of a smartphone by means of an App to implement such a prediction system with a pre-trained neural network to thereby provide a highly portable prediction aid to a clinician. The clinician, taking into account the results of the prediction is then able to apply appropriate therapy to the subject. One such system is described in Porter, P., Abeyratne, U., Swarnkar, V. et al. A prospective multicenter study testing the diagnostic accuracy of an automated cough sound centered analytic system for the identification of common respiratory disorders in children. Respir Res 20, 81 (2019). (herein referred to as the Porter et al paper).

However, it will be realized that determining the values of a number of features such as deviation from Gaussian distribution, log energy level and other computationally intensive features requires complex programming that is technically demanding. Furthermore, it is far from trivial to select an optimal set of features to use to form the feature vectors for a target malady to be diagnosed. Testing, intuition, and flashes of inspiration are often required to arrive at an optimal or near-optimal set of features.

It would be highly advantageous if a method and apparatus for the automatic diagnosis of respiratory maladies from subject sounds was available which was an improvement, or at least a useful alternative, to those of the prior art that have been discussed.

### SUMMARY OF THE INVENTION

According to a first aspect there is provided a method for predicting the presence of a malady of a respiratory system in a subject comprising:
operating at least one electronic processor to transform one or more segments of sounds in an audio recording of the subject, that are associated with the malady, into corresponding one or more image representations of said segments of sounds;
operating the at least one electronic processor to apply said one or more image representations to at least one pattern classifier trained to predict the presence of the malady from the image representations; and
operating the at least one electronic processor (“said processor”) to generate a prediction of the presence of the malady in the subject based on at least one output of the pattern classifier.

In an embodiment the method includes operating said processor to transform the one or more segments of sounds into the corresponding one or more image representations wherein the image representations relate frequency on one axis to time on another axis.

In an embodiment the image representations comprise spectrograms.

In an embodiment the image representations comprise mel-spectrograms.

In an embodiment the method includes operating said processor to identify the potential cough sounds as cough audio segments of the audio recording by using first and second cough sound pattern classifiers trained to respectively detect initial and subsequent phases of cough sounds.

In an embodiment the image representations have a dimension of N x M pixels where the images are formed by said processor processing N windows of each of the segments wherein each window is analyzed in M frequency bins.

In an embodiment each of the N windows overlaps with at least one other of the N windows.

In an embodiment the length of the windows is proportional to length of its associated cough audio segment.

In an embodiment the method includes operating said processor to calculate a Fast Fourier Transform (FFT) and a power value per frequency bin to arrive at a corresponding pixel value of the corresponding image representation of the or more image representations.

n an embodiment the method includes operating said processor to calculate a power value per frequency bin in the form of M power values, being power values for each of the M frequency bins.

n an embodiment the M frequency bins comprise M mel-frequency bins, the method including operating said processor to concatenate and normalize the M power values to thereby produce the corresponding image representation in the form of a mel-spectrogram image.

In an embodiment the image representations are square and M equals N.

In an embodiment the method includes operating said processor to receive input of symptoms and/or clinical signs in respect of the particular malady.

In an embodiment the method includes operating said processor to apply the symptoms and/or clinical signs to the at least one pattern classifier in addition to the one or more image representations.

In an embodiment the method includes operating said processor to predict the presence of the malady in the subject based on the at least one output of the at least one pattern classifier in response to the at least one image representations and the symptoms and/or clinical signs.

In an embodiment the at least one pattern classifier comprises:
a representation pattern classifier responsive to said representations; and
a symptom classifier responsive to said symptoms and/or clinical signs.

In an embodiment the representation pattern classifier comprises a neural network.

In an embodiment the neural network is a convolutional neural network (CNN).

In an embodiment the symptom pattern classifier comprises a logistic regression model (LRM).

In an embodiment the method includes operating said processor to determine a symptom-based prediction probability based on one or more outputs from the symptom pattern classifier.

In an embodiment the method includes operating said processor to determine a representation-based prediction probability based on one or more outputs from the representation pattern classifier.

In an embodiment the method includes determining the representation-based prediction probability based on one or more outputs from the representation pattern classifier in response to between two and seven representations.

In an embodiment the method includes determining the representation-based prediction probability based on one or more outputs from the representation pattern classifier in response to five representations.

In an embodiment the method includes determining the representation-based prediction probability as an average of representation-based prediction probabilities for each representation.

In an embodiment the method includes determining an overall prediction probability value based on the representation-based prediction probability and the symptom-based prediction probability.

In an embodiment the method includes determining the overall probability value as a weighted average of the representation-based probability and the symptom-based probability.

In an embodiment the method includes operating said processor to make a comparison of the representation-based prediction probability value with a predetermined threshold value.

In an embodiment the method includes operating said processor to make a comparison of the overall probability value with a predetermined threshold value.

In an embodiment the method includes operating said processor to present on a display screen responsive to said processor, an indication that the malady is present or is not present based on the comparison.

According to a further aspect there is provided an apparatus for predicting the presence of a respiratory malady in a subject comprising:
an audio capture arrangement configured to store a digital audio recording of a subject in an electronic memory;
a sound segment-to-image representation assembly arranged to transform sound segments of the recording associated with the malady into image representations thereof;
at least one pattern classifier in communication with the sound segment-to-image representation assembly that is configured to process an image representation to produce a signal indicating a probability of the subject sound segment being predictive of the respiratory malady.

In an embodiment the apparatus includes a segment identification assembly in communication with the electronic memory and arranged to process the digital audio recording to thereby identify the segments of the digital audio recording comprising sounds associated with a malady for which a prediction is sought.

In an embodiment the segment identification assembly is arranged to process the digital audio recording to thereby identify the segments of the digital audio recording comprising sounds associated with the malady, wherein the malady comprises pneumonia and the segments comprise cough sounds of the subject.

In an embodiment the segment identification assembly is arranged to process the digital audio recording to thereby identify the segments of the digital audio recording comprising sounds associated with the malady, wherein the malady comprises asthma and the segments comprise wheeze sounds of the subject.

According to a further aspect of the invention there is provided a method for training a pattern classifier to predict the presence of a respiratory malady in a subject from a sound recording of the subject, the method comprising:
transforming sounds associated with the malady, of subjects suffering from and not suffering from the malady, into corresponding image representations;
training the pattern classifier to produce an output predicting presence of the malady in response to application of image representations corresponding to the sounds associated with the malady from subjects suffering from the malady and to produce an output predicting non-presence of the malady in response to application of image representations corresponding to said sounds from subjects not suffering from the malady.

According to a further aspect of the present invention there is provided a method for predicting the presence of a respiratory malady in a subject based on an image representation of a segment of sound from the subject.

According to another aspect of the present invention there is provided an apparatus for predicting the presence of a respirator malady in a subject, the apparatus configured to transform a segment of sound from the subject into a corresponding image representation.

According to another aspect of the present invention there is provided computer readable media bearing tangible, non-transitory machine-readable instructions for one or more processors to implement a method for predicting the presence of a respiratory malady in a subject based on an image representation of a segment of sound from the subject.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is a flowchart of a malady prediction method according to an embodiment of the present invention.

FIG. 2 is a block diagram of a respiratory malady prediction machine.

FIG. 2A is a graph depicting a series of cough sounds and corresponding outputs of first and second trained pattern classifiers.

FIG. 3 is an interface screen display of the machine for eliciting input of a subject's symptoms in respect of the malady.

FIG. 4 is an interface screen display of the machine during recording of sounds of the subject.

FIG. 5 is a diagram illustrating steps in the method that are implemented by the machine to produce image representations of sounds of the subject that are associated with the malady.

FIG. 6 is a Mel-Spectrogram image representation of a subject sound associated with the malady.

FIG. 7 is a Delta Mel-Spectrogram image representation of a subject sound associated with the malady.

FIG. 8 is an interface screen display of the machine for presenting a prediction of the presence of a malady condition in the subject.

FIG. 9 is a block diagram of a convolutional neural network (CNN) training machine according to an embodiment of the invention.

FIG. 10 is a flowchart of a method that is coded as instructions in a software product that is executed by the training machine of FIG. 9.

### DETAILED DESCRIPTION OF PREFERRED EMBODIMENTS

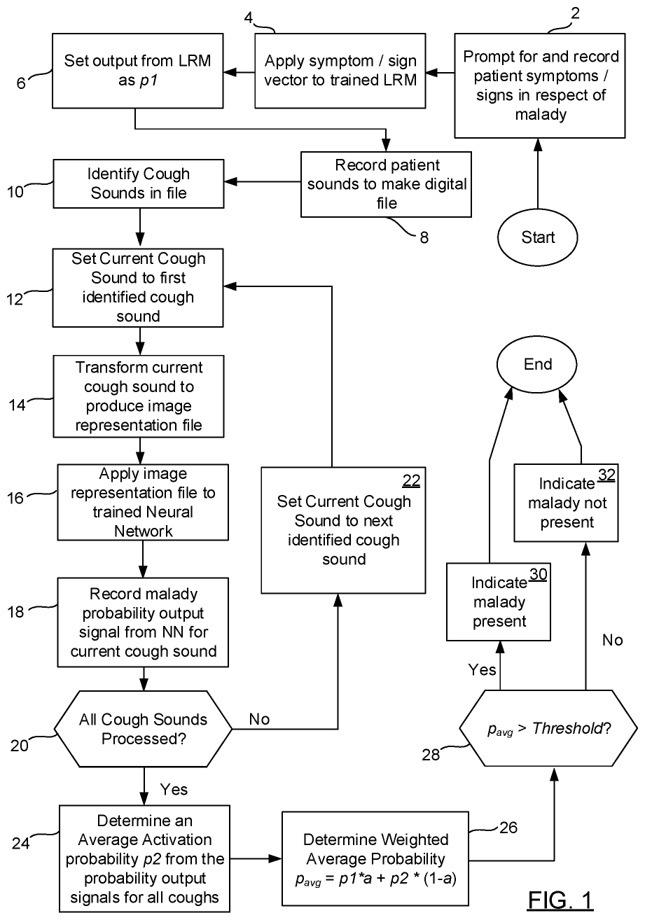

FIG. 1 presents a flowchart of a method according to a preferred embodiment of the present invention for predicting the presence of a malady, such as a respiratory disease in a subject. As will be discussed, the flowchart of FIG. 1 combines a representation-based prediction probability, which is based on image representations of portions of subject sounds, with a symptom-based prediction probability. The symptom-based prediction probability is based on self-assessed subject symptoms in respect of the malady. As will be discussed further, in other embodiments the self-assessed symptoms are not used and the prediction is based only on the image representations of the portions of the subject sounds.

A hardware platform that is configured to implement the method comprises a respiratory malady prediction machine. The machine may be a desktop computer or a portable computational device such as a smartphone that contains at least one processor in communication with an electronic memory that stores instructions that specifically configure the processor in operation to carry out the steps of the method as will be described. It will be appreciated that it is impossible to carry out the method without the specialized hardware, i.e. either a dedicated machine or a machine that is comprised of specially programmed one or more processors. Alternatively, the machine may be implemented as a dedicated assembly that includes specific circuitry to carry out each of the steps that will be discussed. The circuitry may be largely implemented using a Field Programmable Gate Array (FPGA) configured according to a Hardware Descriptor Language (HDL) or Verilog specification.

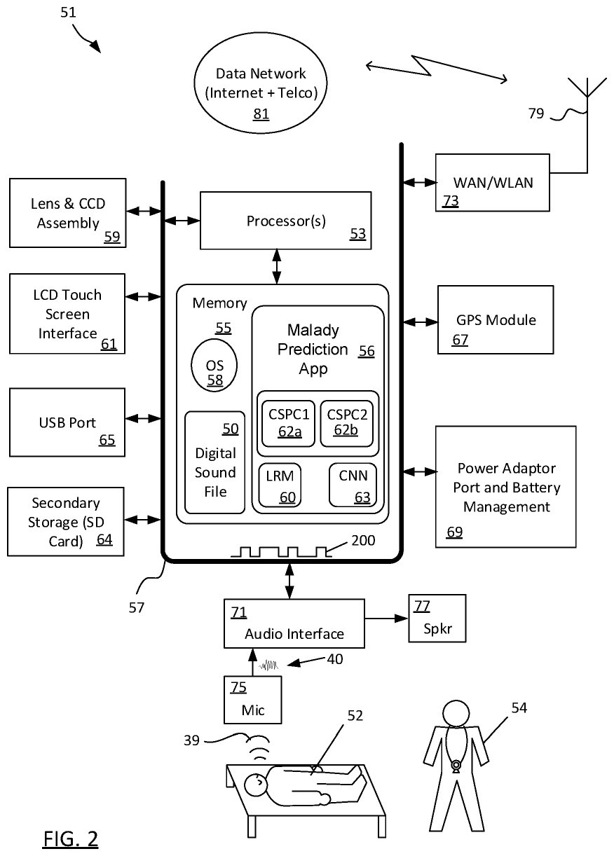

FIG. 2 is a block diagram of an apparatus comprising a respiratory malady prediction machine 51 that, in the presently described embodiment, is implemented using the one or more processors and memory of a smartphone. The respiratory malady prediction machine 51 includes at least one processor 53, which may be referred to as “the processor” for short, that accesses an electronic memory 55. The electronic memory 55 includes an operating system 58 such as the Android operating system or the Apple iOS operating system, for example, for execution by the processor 53. The electronic memory 55 also includes a respiratory malady prediction software product or “App” 56 according to a preferred embodiment of the present invention. The respiratory malady prediction App 56 includes instructions that are executable by the processor 53 in order for the respiratory malady prediction machine 51 to process sounds from a subject 52 and present a prediction of the presence of a respiratory malady in the subject 52 to a clinician 54 by means of LCD touch screen interface 61. The App 56 includes instructions for the processor to implement a pattern classifier such as a trained predictor or decision machine, which in the presently described preferred embodiment of the invention comprises a specially trained Convolutional Neural Network (CNN) 63 and a specially trained Logistic Regression Model (LRM) 60.

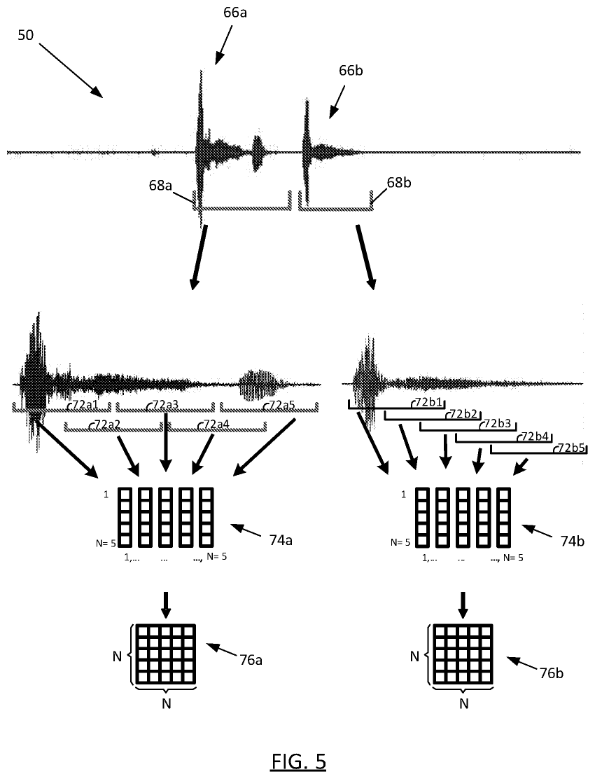

The processor 53 is in data communication with a plurality of peripheral assemblies 59 to 73, as indicated in FIG. 2, via a data bus 57 which is comprised of metal conductors along which digital signals 200 are conveyed between the processor and the various peripherals. Consequently, if required the respiratory malady prediction machine 51 is able to establish voice and data communication with a voice and/or data communications network 81 via WAN/WLAN assembly 73 and radio frequency antenna 79. The machine also includes other peripherals such as Lens & CCD assembly 59 which effects a digital camera so that an image of subject 52 can be captured if desired. A LCD touch screen interface 61 is provided that acts as a human-machine interface and allows the clinician 54 to read results and input commands and data into the machine 51. A USB port 65 is provided for effecting a serial data connection to an external storage device such as a USB stick or for making a cable connection to a data network or external screen and keyboard etc. A secondary storage card 64 is also provided for additional secondary storage if required in addition to internal data storage space facilitated by Memory 55. Audio interface 71 couples a microphone 75 to data bus 57 and includes anti-aliasing filtering circuitry and an Analog-to-Digital sampler to convert the analog electrical waveform from microphone 75 (which corresponds to subject sound wave 39) to a digital audio signal 50 (shown in FIG. 5) that can be stored in memory 55 and processed by processor 53. The audio interface 71 is also coupled to a speaker 77. The audio interface 71 includes a Digital-to-Analog converter for converting digital audio into an analog signal and an audio amplifier that is connected to speaker 71 so that audio recorded in memory 55 or secondary storage 64 can be played back for listening by clinician 54. It will be realized that the microphone 75 and audio interface 71 along with processor 53 programmed with App 56 comprise an audio capture arrangement that is configured for storing a digital audio recording of subject 52 in an electronic memory such as memory 55 or secondary storage 64.

The respiratory malady prediction machine 51 is programmed with App 56 so that it is configured to operate as a machine for classifying subject sound, possibly in combination with subject symptoms, as predictive of the presence a particular respiratory malady in the subject.

As previously discussed, although the respiratory malady prediction machine 51 that is illustrated in FIG. 2 is provided in the form of smartphone hardware that is uniquely configured by App 56 it might equally make use of some other type of computational device such as a desktop computer, laptop, or tablet computational device or even be implemented in a cloud computing environment wherein the hardware comprises a virtual machine that is specially programmed with App 56. Furthermore, a dedicated respiratory malady prediction machine might also be constructed that does not make use of a general purpose processor. For example, such a dedicated machine may have an audio capture arrangement including a microphone and analog-to-digital conversion circuitry configured to store a digital audio recording of the subject in an electronic memory. The machine further includes a segment identification assembly in communication with the memory and arranged to process the digital audio recording to thereby identify segments of the digital audio recording comprising sounds associated with a malady for which a prediction is sought. For example, the malady may comprise pneumonia and the segments may comprise cough sounds of the subject. As another example, the malady may comprise asthma and the segments may comprise wheeze sounds of the subject. A sound segment to image representation assembly may be provided that transforms identified sound segments into image representations. The dedicated machine further includes a hardware implemented pattern classifier in communication with the feature extraction processor that is configured to produce a signal indicating the subject sound segment as being indicative of a respiratory malady.

An embodiment of the procedure that respiratory malady prediction machine 51 uses to predict the presence of a respiratory malady in subject 52, and which comprises instructions that make up App 56 is illustrated in the flowchart of FIG. 1 and will now be described in detail.

At box 2 clinician 54, or another carer or even subject 39, selects App 56 which contains instructions that cause processor 53 to operate LCD Touch Screen Interface 61 to display screen 80 as shown in FIG. 2. The subject's age and the presence and/or severity of symptoms, such as Fever, Wheeze and Cough are then entered and stored in memory 55 as a symptom test feature vector. Clinical signs may also be entered such as the subject's dissolved oxygen level in %, respiratory rate, heart rate etc. Control then proceeds to box 4 of FIG. 1 where the processor 53 applies the symptom test feature vector to a symptom pattern classifier in the form of a pre-trained L2 Regularized Logistic Regression

Model 60 which the App 56 is programmed to implement.

The output from the LRM 60 is a signal, e.g. a digital electrical signal, that indicates the probability of the symptom test feature vector being associated with a particular malady that the subject 52 is suffering from. For example, if the LRM has been pre-trained with training vectors corresponding to people suffering/not suffering from a particular malady, such as pneumonia then the output of the LRM will indicate a probability pi that the subject is suffering from the malady. At box 6 the processor 53 sets the symptom-based prediction probability pi value based on the output from LRM 60.

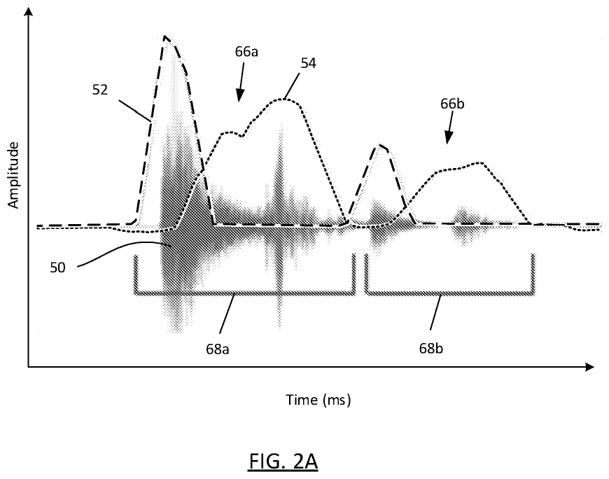

At box 8 the processor 53 displays a screen such as screen 82 of FIG. 3 to prompt the clinician 54 to operate machine 51 to commence recording sound 39 from subject 52 via microphone 75 and audio interface 71. The audio interface 71 converts the sound into digital signals 200 which are conveyed along bus 57 and recorded as a digital file by processor 53 in memory 55 and/or secondary storage SD card 64. In the presently described preferred embodiment the recording should proceed for a duration that is sufficient to include a number of sounds associated with the malady in question to be present in the sound recording.

At box 10 processor 53 identifies segments of the sound that are characterizing of the particular malady. For example, where the malady is pneumonia then the App 56 contains instructions for the processor 53 to process the digital sound file to identify cough sound segments.

A preferred method for identifying cough sounds is described in international patent application publication WO 2018/141013 (sometimes called the “LW2” method herein), the disclosure of which is hereby incorporated herein in its entirety by reference. In the LW2 method feature vectors from the subject sound are applied to two pre-trained neural nets, which have been respectively trained for detecting an initial phase of a cough sound and a subsequent phase of a cough sound. The first neural net is weighted in accordance with positive training to detect the initial, explosive phase, and the second neural net is positively weighted to detect one or more post-explosive phases of the cough sound. In a preferred embodiment of the LW2 method the first neural net is further weighted in accordance with positive training in respect of the explosive phase and negative training in respect of the post-explosive phases. LW2 is particularly good at identifying cough sounds in a series of connected coughs.

At box 10 processor 53 identifies potential cough sounds (PCSs) in the audio sound files 50. In a preferred embodiment of the invention the App 56 includes instructions that configure processor 53 to implement a first cough sound pattern classifier (CSPC1) 62a and a second cough sound pattern classifier (CSPC2) 62b , each preferably comprising neural networks trained to respectively detect initial and subsequent phases of cough sounds. Thus, in the preferred embodiment the processor 53 identifies the PCSs using the LW2 method that has been previously discussed.

Other methods for cough sound detection are also known in the prior art which may also be used. For example, for example, in WO2013/142908 by Abeyratne at al. there is described a method for cough detection which involves determining a number of features for each of a plurality of segments of a subject's sound, forming a feature vector from those features and applying them to a single pre-trained classifier. The output from the classifier is then processed to deem the segments as either “cough” or “non-cough”.

FIG. 2A is a graph showing a portion of the audio recording of sound wave 40 from subject 52. The audio recording is stored as digital sound file 50 in memory 55.

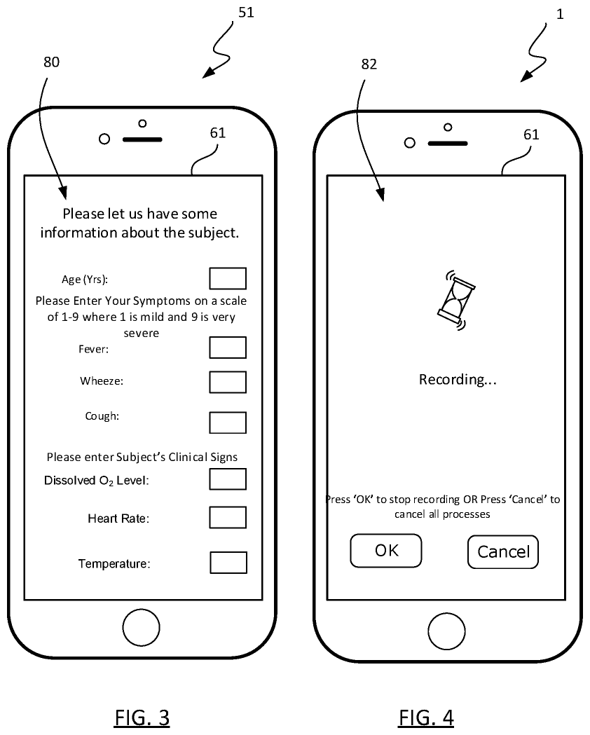

An example of the application of the LW2 method described in WO 2018/141013, which is preferably implemented by processor 53 at box 10, will now be explained. The LW2 method involves applying features of the sound wave to the two trained neural networks CSPC1 62a and CSPC2 62b, which are respectively trained to recognize a first phase and a second phase of a cough sound. The output of the first neural network CSPC1 62a is indicated as line 54 in FIG. 4 and comprises a signal that represents the likelihood of a corresponding portion of the sound wave being a first phase of a cough sound.

The output of the second neural network CSPC2 62b is indicated as line 52 in FIG. 4 and comprises a signal that represents the likelihood of a

WO 2021/119742 PCT/AU2020/051382 corresponding portion of the sound wave being a subsequent phase of the cough sound. Based on the outputs 54 and 52 of the first and second trained neural networks CSPC1 62a and CSPC2 62b , processor 53 identifies two cough sounds 66a and 66b which are located in segments 68a and 68b.

At box 12 the processor sets a variable Current Cough Sound to the first cough sound that has been identified in the sound file.

At box 14 the processor transforms the current cough sound to produce a corresponding image representation which it stores, for example as a file, in either memory 55 or secondary storage 64.

This image representation may comprise, or be based on, a spectrogram of the Current Cough Sound portion of the digital audio file. Possible image representations include mel-frequency spectrogram (or “mel-spectrogram”), continuous wavelet transform, and derivatives of these representations along the time dimension, also known as delta features.

An example of one particular implementation of box 14 is depicted in FIG. 5. Initially the processor 53 identifies two cough sounds 66a , 66b in the digital sound file 50.

Processor 53 identifies the detected coughs 66a and 66b as separate cough audio segments 68a and 68b. Each of the separate cough audio segments 68a and 68b are then divided into N, in the present example N=5, equal length overlapping windows 72a 1,... ,72a 5 and 72b 1,... ,72b 5. For a shorter cough segment, e.g. cough segment 68b which is somewhat shorter than cough segment 68a, the overlapping windows 72b that are used to segment section 68b are proportionally shorter to the overlapping windows 72a that are used to segment section 68a.

Processor 53 then calculates a Fast Fourier Transform (FFT) and a power per mel-bank to arrive at corresponding pixel values. Machine readable instructions for operating a processor to perform these operations on the sound wave are included in App 56. Such instructions are publicly available, for example at: https://librosa.github.io/librosa/_modules/librosa/core/spectrum.html (retrieved 11 December 2019).

In the example illustrated in FIG. 5, processor 53 extracts N=5 Mel-spectrograms 74a , 74b , each with N=5 Mel-frequency bins, from each of the N=5 overlapping windows 72a1, . . . ,72a 5 and 72b 1, . . . ,72b 5.

Processor 53 concatenates and normalizes the values stored in the spectrograms 74a and 74b to produce corresponding Square Mel-Spectrogram images 76a and 76b being image representations representing cough sounds 66a and 66b respectively. Each of images 76a and 76b is an 8-bit greyscale N×N image.

N may be any positive integer value bearing in mind that at some N, depending on the sampling rate of the audio interface 71, the cough image will contain all information present in the original audio, which is desirable. The number of FFT bins may need to be increased to accommodate higher N.

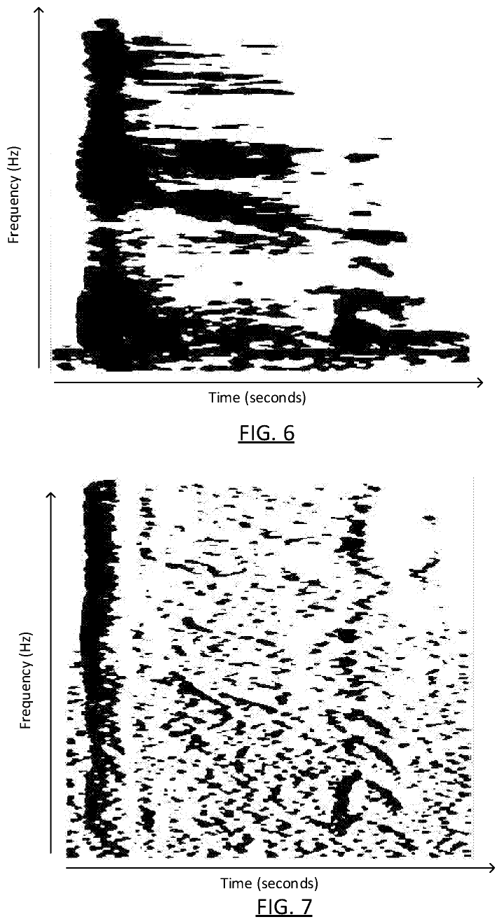

FIG. 6 is a Square Mel-spectrogram image obtained using the process described in FIG. 5 with N=224. In this image, time increases on the horizontal axis from left to right and frequency increases on the vertical axis from bottom to top. Darker areas denote increased amplitude of the mel-frequency bin.

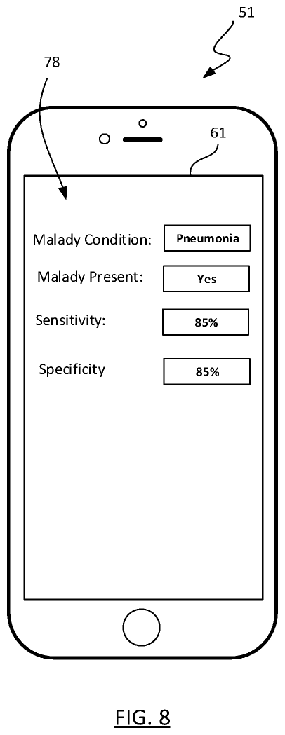

FIG. 7 is a Square Delta Mel-spectrogram image obtained using a process similar to that described in FIG. 5 with N=224. In this image darker areas denote a positive delta and lighter areas a negative delta.

Both FIG. 6 and FIG. 7 have been thresholded so that they are black and white images for purposes of official publication of this patent specification.

Although it is convenient to use square representations that are N×M pixels derived from N×M segments each analyzed for M Mel-frequency bins, where N=M. In other embodiments N may not equal M so that the images that are produced will be square, which is perfectly satisfactory provided that the CNN is trained using similarly dimensioned training images.

From the discussion of box 14 it will be understood that processor 53 configured by App 56 to perform the procedure of box 14 comprises a sound segment-to-image representation assembly that is arranged to transform identified sound segments of the recording, associated with a malady, into corresponding image representations.

Returning now to FIG. 1, at box 16 processor 53 applies the image representation, for example image 76a to a pattern classifier in the form of the trained convolutional neural network (CNN) 63. The CNN 63 is trained to predict the presence of a particular respiratory malady in the subject 52 from the image 76a . The CNN 63 comprises a pattern classifier that generates a prediction of the presence of the malady in the form of an output probability signal. The output probability signal ranges between 0 and 1 wherein 1 indicates a certainty that the malady is present in the subject and 0 indicates that there is no likelihood of the malady being present. Processor 53 records a representation-based prediction probability for the image representation for the current cough sound. At box 20 a check is performed and if there are more coughs to be processed then control diverts back to box 12 and the process is repeated. Alternatively, if at box 20 all cough sounds have been processed then control proceeds to box 24.

It will be realized that the CNN 63 comprises a pattern classifier that is configured to generate an output indicating a probability of the subject sound segment being predictive of the respiratory malady.

At box 24 the processor 53 determines an average activation probability p₂ from the probability output signals for all of the coughs. At box 26 the processor 53 combines the probability of the respiratory malady being present pi, which is based on the subject's symptoms, with the average activation probability p₂ that is the representation-based probability prediction that has been determined from the output of the CNN in response to the images. The p_{avg} probability that is determined at box 26 is the weighted average of p₁ and p₂, weighted by a factor “a”. The factor “a” is typically 0.5.

At box 28 the processor 53 compares the p_{avg} value to a predetermined Threshold value. How the Threshold value is determined will be described later. If p_{avg} is greater than Threshold then processor 53 indicates whether or not the respiratory malady in question is indicated to be present. In the presently described embodiment processor 53 operates LCD Touch Screen Interface 61 to display the screen 78 shown in FIG. 8. Screen 78 presents the name of the malady that has been detected (e.g. “Pneumonia”) and whether or not it has been determined to be present.

In other embodiments of the invention the processor 53 does not collect subject symptoms and/or clinical signs and so does not perform boxes 2, 4, 6 and 26. Instead at box 28 p2 is compared to the Threshold and the indications of whether or not a malady are present that are made at boxes 30 and 32 are made on the basis of p₂ only.

Performance

The performance of the diagnosis methods described in the previously referred to Porter et al. paper was compared to various embodiments of the present invention.

A study recruited 1021 subjects from Joondalup Health Campus in Perth, Western Australia. The subjects were recruited from an acute general hospital ED, wards, and outpatient clinics. The performance of the diagnosis methods was evaluated using sensitivity and specificity compared to a clinical diagnosis reached by expert clinicians with full examination and results of investigation. The demographics of the set are as following. The set has 628 females and 393 males. The median female age is 67 years, with minimum age of 16 and maximum 99. Median male age is 68 years, minimum 16 and maximum 93 years.

The results were pooled on the whole data set using a 25-fold cross-validation method. Both results for the old method and the method of the embodiment described herein were 25-fold cross validations on the same data set. The model building was done only using the subjects in the training folds only. The training was done using all the coughs in each recording. However, in the validation the Inventors used only the first five coughs because that is the preferred number of coughs to use in the procedures that have been discussed with reference to FIG. 1, i.e. box 20 diverts to box 24 after five coughs have been processed in boxes 12 to 18.

Table 1 compares the prior art procedure that is the subject of the Porter et al. paper with the previously mentioned embodiment of the present invention in which the processor 53 does not collect subject symptoms and so does not perform boxes 2, 4, 6 and 26 of FIG. 1. Instead at box 28 p₂ is compared to the Threshold and the indications of whether or not a malady are present that are made at boxes 30 and 32 are made on the basis of p₂ only.

TABLE 1
performance of the two cough diagnosis algorithms
on the adult respiratory disease cohort

| | Diagnosis algorithm described | | Procedure according to | |
|---|---|---|---|---|
| | in Porter et al. without use of | | FIG. 1 without use of | |
| | subject signs. | | subject symptoms. | |
| | Sensitivity | Specificity | Sensitivity | Specificity |
| | (%) | (%) | (%) | (%) |
| ASTHMA_EX | 75.9 | 73.7 | 79.7 | 87.4 |
| COPD | 65.7 | 76.9 | 78.5 | 84.6 |
| COPD_EX | 76.2 | 69.5 | 76.2 | 84.6 |
| LRTD | 79.2 | 76.9 | 87.7 | 77.7 |
| PNEUMONIA | 74.2 | 74.6 | 81.3 | 80.0 |

Table 2 compares the performance of the diagnosis procedure described in Porter et al. including supplementation by use of subject signs with the embodiment of the present invention described with reference to FIG. 1.

Cough Sound and Clinical Symptoms Ensemble

TABLE 2
performance of the two cough and signs diagnosis algorithms
on the adult respiratory disease cohort

| | Diagnosis algorithm | | Ensemble of | |
|---|---|---|---|---|
| | described in Porter | | Representation- | |
| | et al. with use of | | based and and Symptom- | |
| | subject symptoms. | | based CNN and LRM outputs | |
| | Sensitivity | Specificity | Sensitivity | Specificity |
| | (%) | (%) | (%) | (%) |
| ASTHMA_EX | 88.6 | 82.1 | 82.3 | 89.5 |
| COPD | 84.3 | 85.5 | 88.1 | 90.9 |
| COPD_EX | 85.7 | 85.4 | 88.4 | 81.7 |
| LRTD | 86.4 | 84.6 | 90.6 | 84.6 |
| PNEUMONIA | 86.9 | 85.4 | 89.7 | 86.2 |

It will be observed from Table 1 and Table 2 that procedures according to embodiments of the present invention result in improved performance of the diagnosis. More importantly though, the embodiments according to the present invention avoid the need to hand-craft audio features and construct sophisticated classification systems manually.

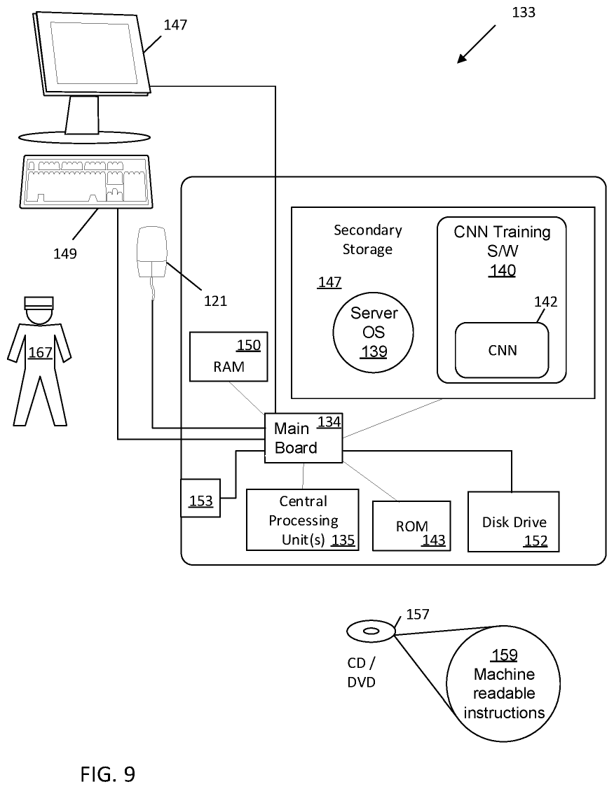

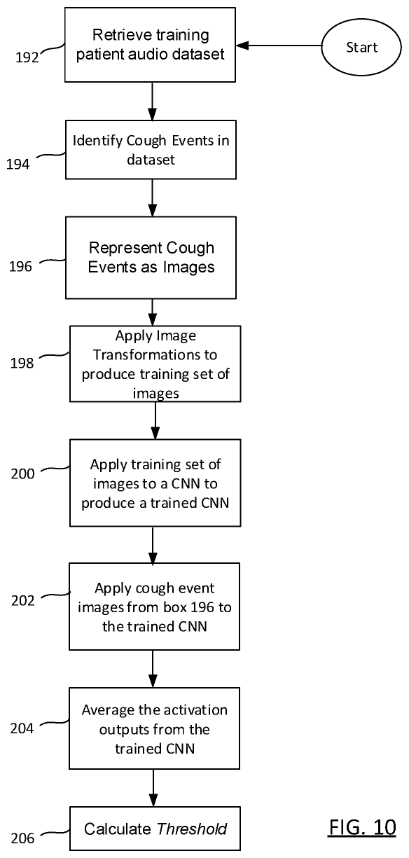

FIG. 9 is a block diagram of a CNN training machine 133 implemented using the one or more processors and memory of a desktop computer configured according to CNN training Software 140. CNN training machine 133 includes a main board 134 which includes circuitry for powering and interfacing to one or more onboard microprocessors 135.

The main board 134 acts as an interface between microprocessors 135 and secondary memory 147. The secondary memory 147 may comprise one or more optical or magnetic, or solid state, drives. The secondary memory 147 stores instructions for an operating system 139. The main board 134 also communicates with random access memory (RAM) 150 and read only memory (ROM) 143. The ROM 143 typically stores instructions for a startup routine, such as a Basic Input Output System (BIOS) or Unified Extensible Firmware Interface (UEFI) which the microprocessor 135 accesses upon start up and which preps the microprocessor 135 for loading of the operating system 139.

The main board 134 also includes an integrated graphics adapter for driving display 147. The main board 133 will typically include a communications adapter 153, for example a LAN adaptor or a modem or a serial or parallel port, that places the server 133 in data communication with a data network.

An operator 167 of CNN training machine 133 interfaces with it by means of keyboard 149, mouse 121 and display 147.

The operator 167 may operate the operating system 139 to load software product 140. The software product 140 may be provided as tangible, non-transitory, machine readable instructions 159 borne upon a computer readable media such as optical disk 157. Alternatively it might also be downloaded via port 153.

The secondary storage 147, is typically implemented by a magnetic or solid state data drive and stores the operating system, for example Microsoft Windows, and Ubuntu Linux Desktop are two examples of such an operating system.

The secondary storage 147 also includes software product 140, being a CNN training software product 140 according to an embodiment of the present invention. The CNN training software product 140 is comprised of instructions for CPUs 135 (or as alternatively and collectively referred to “processor 135”) to implement the method that is illustrated in FIG. 10.

Initially at box 192 of FIG. 10 processor 135 retrieves a training subject audio dataset which will typically be comprised of a number of files containing subject audio and metadata from a data storage source via communication port 153. The metadata includes training labels, i.e. information about the subject, e.g. age, gender etc and whether or not the subject suffers from each of a number of respiratory maladies.

At box 194 segments of audio, such as coughs in respect of pneumonia, or other sounds, for example wheeze sounds in respect of asthma, associated with a particular malady are identified. The cough events in the data for each subject are identified, for example in the same manner as has previously been discussed at box 10 of FIG. 1.

At box 196 the processor 135 represents the cough events as images in the same manner as has previously been discussed at box 14 of FIG. 1 wherein Mel-spectrogram images are created to represent each cough.

At box 198 processor 135 transforms each Mel-spectrogram to create additional training examples for subsequently training a convolutional neural net (CNN). This data augmentation step is preferable because the CNN is a very powerful learner and with limited number of training images it can memorize the training examples and thus over fit the model. The Inventors have discerned that such a model will not generalize well on previously unseen data. The applied image transformations include, but are not limited to, small random zooming, cropping and contrast variations.

At box 200 the processor 135 trains the CNN 142 on the augmented cough images that have been produced at box 198 and the original training labels. Over fitting of the CNN is further reduced by using regularization techniques such as dropout, weight decay and batch normalization.

One example of the process used to produce a CNN is to take a pretrained ResNet model, which is a residual network containing shortcut connections, such as ResNet-18, and use the convolutional layers of the model as a backbone, and replace the final non-convolutional layers with layers that suit this problem domain. These include fully connected hidden layers, dropout layers and batch normalization layers. Information about ResNet-18 is available at https://www.mathworks.com/help/deeplearning/ref/resnet18.html (retrieved 2 December 2010), the disclosure of which is incorporated herein by reference.

ResNet-18 is a convolutional neural network that is trained on more than a million images from the ImageNet database (http://www.image-net.org). The network is 18 layers deep and can classify images into 1000 object categories, such as keyboard, mouse, pencil, and many animals. As a result, the network has learned rich feature representations for a wide range of images. The network has an image input size of 224-by-224.

The Inventors have found that it is sufficient to fix the ResNet-18 layers and only train the new non-convolutional layers, however it is also possible to re-train both the ResNet-18 layers and the new non-convolutional layers to achieve a working model. A fixed dropout ratio of 0.5 is preferably used. Adaptive Moment Estimation (ADAM) is preferably used as an adaptive optimizer though other optimizer technique may also be used.

At box 202 the original (non-augmented) cough images from box 196 are applied to the CNN 142 which is now trained to elicit probabilities for each cough indicating a particular malady from the trained CNN 142.

At box 204 processor 135 calculates the average probability of each recording's cough and deems it a per-recording activation.

At box 206 the per-recording activation is used to calculate the Threshold value which provides the desired performance characteristics and which is used at box 28 of FIG. 1.

The trained CNN is then distributed as CNN 63 as part of Malady Prediction App 56.

To recap, in one aspect there is provided a method for predicting the presence of a malady, for example but not limited to pneumonia or asthma, of a respiratory system in a subject 52. The method involves operating at least one electronic processor 53 to transform one or more segments e.g. segments 68a, 68b of sounds 40 in an audio recording such as as digital sound file 50, of the subject, that are associated with the malady, into corresponding one or more image representations such as representations 74a, 74b and 76a, 76b. The method also involves operating the at least one electronic processor 53 to apply the one or more image representations, e.g. representations 76a, 76b, to at least one pattern classifier 63 that has been trained to predict the presence of the malady from the image representations. The method also involves operating the at least one electronic processor 53 to generate a prediction (boxes 30 and 32 of FIG. 1) of the presence of the malady in the subject based on at least one output (box 18 of FIG. 1) of the pattern classifier 63. For example the prediction may be presented on a screen such as screen 78 (FIG. 8).

In another aspect an apparatus is provided for predicting the presence of a respiratory malady in a subject such as, but not limited to, pneumonia or asthma. The apparatus includes an audio capture arrangement, for example microphone 75 and audio interface 71 along with processor 53 configured by instructions of App 56 to store a digital audio recording of subject 52 in an electronic memory such as memory 55 or secondary storage 64. A sound segment-to-image representation assembly is provided, for example by processor 53, configured by App 56, to perform the procedure of box 14 (FIG. 1) to transform identified sound segments, e.g., segments 68a, 68b, of the recording, such as digital sound file 50, associated with a malady, into corresponding image representations, such as image representations 76a, 76b. The apparatus also includes at least one pattern classifier, for example image pattern classifier 63, that is in communication with the sound segment-to-image representation assembly and which is that is configured, for example by pre-training, to process an image representation to produce a signal indicating a probability of the subject sound segment being predictive of the respiratory malady.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

Throughout the specification and claims (if present), unless the context requires otherwise, the term “substantially” or “about” will be understood to not be limited to the value for the range qualified by the terms.

Any embodiment of the invention is meant to be illustrative only and is not meant to be limiting to the invention. Therefore, it should be appreciated that various other changes and modifications can be made to any embodiment described without departing from the scope of the invention.

1. A method for predicting the presence of a malady of a respiratory system in a subject comprising:
operating at least one electronic processor to transform one or more segments of sounds in an audio recording of the subject, that are associated with the malady, into corresponding one or more image representations of said segments of sounds;
operating the at least one electronic processor to apply said one or more image representations to at least one pattern classifier trained to predict the presence of the malady from the image representations; and
operating the at least one electronic processor to generate a prediction of the presence of the malady in the subject based on at least one output of the pattern classifier.

2. The method of claim 1, including operating said processor the at least one electronic processor to transform the one or more segments of sounds into the corresponding one or more image representations wherein the image representations relate frequency to time.

3. The method of claim 2, wherein the image representations comprise spectrograms or mel-spectrograms.

4. (canceled)

5. The method of claim 1, including operating the at least one electronic processor to identify the potential cough sounds as cough audio segments of the audio recording by using first and second cough sound pattern classifiers trained to respectively detect initial and subsequent phases of cough sounds.

6. The method of claim 1, wherein the image representations have a dimension of N×M pixels where the images are formed by the at least one electronic processor processing N windows of each of the segments wherein each window is analyzed in M frequency bins.

7. The method of claim 6, wherein each of the N windows overlaps with at least one other of the N windows and wherein lengths of the windows are proportional to lengths of their associated cough audio segments.

8. (canceled)

9. The method of claim g7, including operating the at least one electronic processor to calculate a Fast Fourier Transform (FFT) and a power value per frequency bin to arrive at a corresponding pixel value of the corresponding image representation of the or more image representations.

10. The method of claim 9, including operating the at least one electronic processor to calculate a power value per frequency bin in the form of M power values, being power values for each of the M frequency bins.

11. The method of claim 10, wherein the M frequency bins comprise M mel-frequency bins, the method including operating the at least one electronic processor to concatenate and normalize the M power values to thereby produce the corresponding image representation in the form of a mel-spectrogram image.

12. The method of claim 6, wherein the image representations are square and wherein M equals N.

13. The method of claim 1, including operating the at least one electronic processor to receive input of symptoms and/or clinical signs in respect of the malady.

14. The method of claim 13, including operating the at least one electronic processor to apply the symptoms and/or clinical signs to the at least one pattern classifier in addition to the one or more image representations and operating the at least one electronic processor to predict the presence of the malady in the subject based on the at least one output of the at least one pattern classifier in response to the at least one image representations and the symptoms and/or clinical signs.

15. (canceled)

16. The method of claim 14, wherein the at least one pattern classifier comprises:
a representation pattern classifier responsive to said representations; and
a symptom classifier responsive to said symptoms and/or clinical signs.

17.-20. (canceled)

21. The method of claim 16, including operating the at least one electronic processor to determine a representation-based prediction probability based on one or more outputs from the representation pattern classifier.

22. The method of claim 21, including determining the representation-based prediction probability based on one or more outputs from the representation pattern classifier in respond to between two and seven representations.

23. The method of claim 22, including determining the representation-based prediction probability based on one or more outputs from the representation pattern classifier in response to five representations.

24. The method of claim 22, including determining the representation-based prediction probability as an average of representation-based prediction probabilities for each representation.

25.-29. (canceled)

30. An apparatus for predicting the presence of a respiratory malady in a subject comprising:
an audio capture arrangement configured to store a digital audio recording of a subject in an electronic memory;
a sound segment-to-image representation assembly arranged to transform sound segments of the recording associated with the malady into image representations thereof; and
at least one pattern classifier in communication with the sound segment-to-image representation assembly that is configured to process an image representation to produce a signal indicating a probability of the subject sound segment being predictive of the respiratory malady.

31. The apparatus of claim 30, wherein the apparatus includes a segment identification assembly in communication with the electronic memory and arranged to process the digital audio recording to thereby identify the segments of the digital audio recording comprising sounds associated with a malady for which a prediction is sought.

32. The apparatus of claim 31, wherein the segment identification assembly is arranged to process the digital audio recording to thereby identify the segments of the digital audio recording comprising sounds associated with the malady, wherein the malady comprises pneumonia and the segments comprise cough sounds of the subject or the malady comprises asthma and the segments comprise wheeze sounds of the subject.

33. (canceled)
